(19) United States
(12) Patent Application Publication
(10) Pub. No.: US 2025/0061977 A1
(43) Pub. Date: Feb. 20, 2025

(54) SYSTEM AND METHOD FOR USING PHYSICS-BASED DEPICTIONS OF PROTEIN SHAPES IN VISUALIZATION AND SHAPE-CONDITIONED DRUG CANDIDATE GENERATION

(71) Applicant: QuanMol Tech, Inc., Hillsborough, CA (US)

(72) Inventors: Minzhen Yi, Hillsborough, CA (US); Jie Li, San Carlos, CA (US); Bo Li, San Carlos, CA (US); Jieyu Lu, San Carlos, CA (US); Xudong Lv, Pasadena, CA (US); Xingyu Shen, San Carlos, CA (US)

(21) Appl. No.: 18/449,652

(22) Filed: Aug. 14, 2023

(51) Int. Cl. G16C 20/50; G06T 17/20; G16C 20/80

(52) U.S. Cl. CPC G16C 20/50; G06T 17/20; G16C 20/80; G06T 2210/56

(57) ABSTRACT

This disclosure presents a method and system aimed at improving the shape complementarity between pockets and ligands. The method involves several steps, such as determining non-polar interactions among atoms within each region of a molecule, creating point clouds to represent these regions, and generating a mesh that overlays the molecular structure. This mesh enables users to make adjustments to the shape of a ligand, which is intended to enhance the compatibility with the pockets. Additionally, the method provides a visualization of the mesh on the molecular structure, allowing users to observe the precise locations of the regions and the potential fields associated with them.

### TECHNICAL FIELD

The disclosure relates generally to generating physics-based representations of pertinent nonbonded interactions between atoms in the pocket regions within a molecule to guide drug generation or modification.

### BACKGROUND

The maximization of pocket-ligand shape complementarity stands as a primary driving force behind noncovalent protein-ligand binding, exerting a crucial influence on the field of small-molecule drug design. Skillful manipulation of molecular shape and nonpolar interactions holds the potential to rapidly enhance drug potency. Despite its significance, effectively incorporating shape constraints into the drug design process remains a daunting task for medicinal chemists. This challenge primarily stems from the dearth of rapid, atomically-precise, and chemically-intuitive protocols for analyzing pocket shapes, impeding the efficient utilization of shape-based strategies in drug discovery endeavors.

This application describes a system that enables synergistic and simultaneous visualization and molecular generation by utilizing physics-based representations of pertinent nonbonded interactions between atoms in the pocket regions within a molecule. This system relies on a suite of foundational analytical methods, which provide atomically precise representations of nonbonded interactions in three-dimensional space. These analytical methods are intricately integrated with downstream tasks, equipping the computer with a wealth of information regarding shape constraints. As a result, the disclosed system facilitates a comprehensive understanding of the underlying molecular properties, enabling efficient exploration and manipulation of chemical space in the context of drug design.

### SUMMARY

Various embodiments of the specification include, but are not limited to, systems, methods, and non-transitory computer-readable media for generating physics-based representations of pertinent nonbonded interactions between atoms in the pocket regions within a molecule to guide drug generation or modification.

In some aspects, the techniques described herein relate to a computer-implemented method, including: selecting a plurality of regions on a molecule, each region including a plurality of atoms; within each of the plurality of regions, computing nonbonded interactions between each pair of the plurality of atoms in the region to obtain a plurality of physical attributes of the region, each of the plurality of physical attributes representing an interaction strength between the pair of atoms; constructing a point cloud for each of the plurality of regions based on the plurality of physical attributes of the region; obtaining a drug candidate for bonding to a target region in the plurality of regions; and performing molecule modification on the drug candidate to match a shape of the point cloud of the target region.

In some aspects, the method may further including displaying, on a graphic user interface (GUI), a structure of the molecule by overlaying mesh on the plurality of regions based on the point cloud for each of the plurality of regions.

In some aspects, the displaying includes, for each of the plurality of regions on the molecule: performing quantization on the plurality of attributes of the region into a plurality of strength values; and generating a mesh for the region that includes a plurality of vertices corresponding to the plurality of attributes, wherein coordinates of the plurality of vertices are determined based on the plurality of strength values.

In some aspects, the displaying includes, for each of the plurality of regions on the molecule: performing quantization on the plurality of attributes of the region into a plurality of strength values; and generating a mesh for the region by defining color gradients on a face of the mesh based on the plurality of strength values.

In some aspects, the plurality of physical attributes include: quantified repulsion; quantified dispersion attractions, quantified solvation; or quantified hydrophobic effect.

In some aspects, the computing nonbonded interactions between each pair of atoms in the region uses an Lennard-Jones potential.

In some aspects, the computing nonbonded interactions between each pair of atoms in the region to obtain a plurality of physical attributes of the region includes: determining atom types of the pair of atoms based on each atom's chemical properties; determining a first parameter characterizing a strength of an attractive interaction between the pair of atoms; determining a second parameter representing a distance at which a repulsive interaction between the pair of atoms is dominant; and computing a potential interaction strength between the pair of atoms using the first parameter and the second parameter.

In some aspects, the molecule modification for the drug candidate includes: scaffold modification; side chain optimization; conformational sampling; functional group addition or removal; or linker optimization.

In some aspects, the method may further include: determining a non-polar potential field for the region based on the nonbonded interactions between each pair of the plurality of atoms in the region; wherein the constructing the point cloud for the region includes: in response to the non-polar potential field being negative and having an absolute value greater than a threshold, constructing the point cloud for the region.

In some aspects, the techniques described herein relate to a computing system, including: one or more processors; and a memory storing instructions that, when executed by the one or more processors, cause the system to perform operations including: selecting a plurality of regions on a molecule, each region including a plurality of atoms; within each of the plurality of regions, computing nonbonded interactions between each pair of the plurality of atoms in the region to obtain a plurality of physical attributes of the region, each of the plurality of physical attributes representing an interaction strength between the pair of atoms; constructing a point cloud for each of the plurality of regions based on the plurality of physical attributes of the region; obtaining a drug candidate for bonding to a target region in the plurality of regions; and performing molecule modification on the drug candidate to match a shape of the point cloud of the target region.

In some aspects, the techniques described herein relate to a non-transitory computer-readable storage medium configured with instructions executable by one or more processors to cause the one or more processors to perform operations including: selecting a plurality of regions on a molecule, each region including a plurality of atoms; within each of the plurality of regions, computing nonbonded interactions between each pair of the plurality of atoms in the region to obtain a plurality of physical attributes of the region, each of the plurality of physical attributes representing an interaction strength between the pair of atoms; constructing a point cloud for each of the plurality of regions based on the plurality of physical attributes of the region; obtaining a drug candidate for bonding to a target region in the plurality of regions; and performing molecule modification on the drug candidate to match a shape of the point cloud of the target region.

In some aspects, the techniques described herein relate to a non-transitory computer-readable storage medium, wherein the operations further include: determining a non-polar potential field for the region based on the nonbonded interactions between each pair of the plurality of atoms in the region; wherein the constructing the point cloud for the region includes: in response to the non-polar potential field being negative and having an absolute value greater than a threshold, constructing the point cloud for the region.

In some aspects, the techniques described herein relate to a non-transitory computer-readable storage medium, wherein the operations further include: displaying, on a graphic user interface (GUI), a structure of the molecule by overlaying mesh on the plurality of regions based on the point cloud for each of the plurality of regions, wherein the displaying includes: performing quantization on the plurality of attributes of the region into a plurality of strength values; and generating a mesh for the region by assigning the plurality of strength values to corresponding vertices of the mesh.

These and other features of the systems, methods, and non-transitory computer-readable media disclosed herein, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for purposes of illustration and description only and are not intended as a definition of the limits of the invention. It is to be understood that the foregoing general description and the following detailed description are exemplary and explanatory only, and are not restrictive of the invention, as claimed.

### BRIEF DESCRIPTION OF THE DRAWINGS

Preferred and non-limiting embodiments of the invention may be more readily understood by referring to the accompanying drawings in which:

FIG. 1 illustrates an example system diagram for using physics-based depictions of protein shapes in visualization and shape-conditioned drug candidate generation, in accordance with various embodiments.

FIG. 2 illustrates an example method for using physics-based depictions of protein shapes in visualization and shape-conditioned drug candidate generation, in accordance with various embodiments.

FIG. 3 illustrates an example screenshot of the display of a mesh overlaying a protein structure to visualize shape-preconditioned interaction, in accordance with various embodiments.

FIG. 4 illustrates a block diagram of an example computer system in which any of the embodiments described herein may be implemented.

### DETAILED DESCRIPTION OF THE EMBODIMENTS

Specific, non-limiting embodiments of the present invention will now be described with reference to the drawings. It should be understood that particular features and aspects of any embodiment disclosed herein may be used and/or combined with particular features and aspects of any other embodiment disclosed herein. It should also be understood that such embodiments are by way of example and are merely illustrative of a small number of embodiments within the scope of the present invention. Various changes and modifications obvious to one skilled in the art to which the present invention pertains are deemed to be within the spirit, scope, and contemplation of the present invention as further defined in the appended claims.

In order to grasp the technical advantages of the solution described here, it is important to have an understanding of the prevailing approach in the field of drug discovery, molecular design, molecular simulation, computational chemistry, and partially, machine learning and geometric deep learning.

A commonly employed method for drug candidate generation involves using sizable collections of chemical compounds known as compound libraries. These libraries usually include a large collection of diverse chemical compounds that can be screened to identify potential drug candidates. In the traditional drug discovery process, the first step is to identify a specific target, such as a protein or receptor, that is associated with a particular disease or condition. Once a specific target is identified, the next step involves selecting a suitable compound library. The choice of the library depends on several factors, such as the target's characteristics, the desired mechanism of action, and the chemical diversity needed to explore different molecular interactions. Researchers may also access commercially available compound libraries or create custom libraries based on their specific requirements. An iterative screening process is then performed to test a large number of compounds against the target. This process is generally lengthy and inefficient.

During the iterative screening, various computational or experimental techniques are employed to evaluate the compounds based on specific criteria. One important aspect of this process is the use of scoring functions. In general, compound scoring can be obtained from approximate static structure-based models or more comprehensive molecular simulation-based protocols (e.g., free-energy perturbation (FEP)) that estimate the affinity or likelihood of a compound binding to the target (e.g., a protein receptor) or possessing certain properties.

The scoring functions are designed to predict the binding affinity, stability, or other relevant properties of the compounds. These functions consider factors such as molecular shape, electrostatic interactions, hydrogen bonding, hydrophobicity, and other physicochemical properties. The compounds are typically ranked or scored based on their predicted activity or fitness for the target.

However, despite the extensive use of scoring functions, it is challenging to develop a scoring function that is universally effective and accurate for all scenarios. The effectiveness of a scoring function depends on the specific target, the chemical properties of the compounds, and the nature of the interactions involved. Achieving high accuracy in predicting compound activities or properties remains a significant challenge in the field.

In summary, generating compound libraries and relying on scoring functions for iterative screening can be a slow process, and there is no scoring function that is considered sufficiently effective in all cases. Consequently, the success rate of virtual screening remains at a low level of typically 5-10%.

To address these technical challenges, the method and system described herein utilize local point-cloud depictions of pocket shapes to guide molecular modification/growth (e.g., shape-preconditioning the drug molecule). By utilizing the local point cloud, the system can guide the modification or growth of molecules in a more targeted and streamlined manner. This approach avoids the need for the extensive generation of compound libraries and the subsequent iterative screening process.

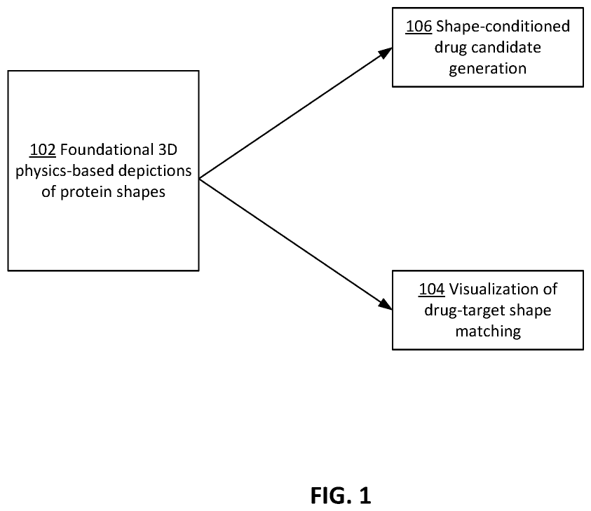

FIG. 1 illustrates an example system diagram for using physics-based depictions of protein shapes in visualization and shape-conditioned drug candidate generation, in accordance with various embodiments. The multiple components in FIG. 1 are for illustrative purposes. Depending on the implementation, the system may include more, fewer, or alternative components.

In some embodiments, the system in FIG. 1 may include a backend module 102 for computing the 3D physics-based depictions of protein shapes, a first downstream module 104 for generating visualization of drug-target shape matching, and a second downstream module 106 for guiding shape-conditioned drug candidate modification and generation.

The backend module 102 can be implemented on a server device or a cloud service, while the downstream modules 104 and 106 may include user-interfaces that interact with researchers. The user-interfaces can take the form of either a desktop application or a web-based application. For example, the user-interface might involve a desktop application installed directly on a user's computer or a web-based application accessed through a web browser. The back-end module 102 is responsible for sending computed data to the desktop application or the web-based application via internet connections. This allows the desktop application or the web-based application to generate visualizations of drug-target shape matching and/or provide guidance in the modification and generation of drug candidates.

In some embodiments, the backend module 102 computes physics-based representations of pertinent nonbonded interactions, including quantified repulsion, quantified dispersion attractions, quantified solvation, or quantified hydrophobic effect (e.g., Pauli repulsion, London dispersion attractions, solvation and hydrophobic effects, etc.) among the atoms of a protein. In molecular interactions, nonbonded interactions refer to the forces or interactions between atoms that are not involved in chemical bonds. In other words, nonbonded interactions occur between particles/atoms that are not connected by chemical bonds but still exert forces on each other due to various physical phenomena. These interactions are also known as intermolecular forces or van der Waals forces. These nonbonded interactions are then used as attributes in constructing a point cloud object overlaying the protein structure, effectively capturing the landscape of potential nonbonded interactions between drug and target molecules.

In some embodiments, the nonbonded interactions may be computed using a non-bond potential function, which typically depends on the types, relative positions, and orientations of the atoms involved. It is often represented as a mathematical equation that quantifies the potential energy of a region (e.g., a pocket area) as a function of these variables. The specific form of the non-bond potential function depends on the type of interaction being considered and the specific model or theory being used. For example, the non-bond potential function may include terms for electrostatic interactions (e.g., Coulombic interactions) and van der Waals interactions (e.g., Lennard-Jones potential). These functions may be designed to capture the forces and energy associated with non-bonded interactions between the atoms in a pocket of the protein or a target molecule.

Once the backend module 102 computes the nonbonded interaction strengths among the atoms, it may generate a point cloud object for each pocket on the protein or the target molecule based on the nonbonded interaction strengths. For example, the nonbonded interaction strengths may first go through a quantization process and be converted into a plurality of quantized strength values. The quantization process is designed to discretize or represent the continuous values of the nonbonded interactions in a discrete form. This quantization process aids in establishing machine-readable interaction encodings that can be subsequently used in machine-learning-based preconditioning options.

The backend module 102 may then generate a point cloud mesh around the pockets of the protein or target molecule based on the corresponding strength values. For example, each vertex of the point cloud may correspond to a nonbond interaction between a pair of atoms in a pocket, and thus be assigned with the corresponding interaction strength value. The coordinates of the vertices in the point cloud mesh may be determined based on the corresponding strength values. This way, a more expansive point cloud overlaying a pocket may indicate a stronger interaction strength, and vice versa. As another example, the point cloud mesh may define color gradients on a face of the mesh based on the plurality of strength values, e.g., different colors may indicate different strength values. As yet another example, the strength values of the nonbonded interactions can be represented through color mapping in the mesh, where stronger interactions are depicted using more intense or vibrant colors, while weaker interactions are represented with less intense or muted colors.

The point cloud-based representation of the protein or the target molecule may be consumed in the downstream modules 106 and 014. For example, in the shape-conditioned drug candidate generation module 106, the point-cloud-based representations of target shapes (e.g., the pockets) may be used to provide precise information about the three-dimensional space where a drug candidate (e.g., a ligand or a molecule from the drug side) can bind and interact with the target. This approach empowers the shape-conditioned drug candidate generation by utilizing the local point-cloud depictions and performing molecular modifications that precisely match the shape of the point cloud.

A significant advantage of this approach is to incorporate prior conditions (the nonbonded interaction strengths represented as point clouds) without the need for post-generation screening. In other words, this point-cloud representation-based solution circumvents the traditional reliance on compound library screening by directly generating new molecules based on shape pre-conditioning. In some embodiments, the shape-conditioned drug candidate generation module 106 may incorporate both manual-defined (e.g., length, width, planarity, sphericity) and machine-learned descriptors for preconditioning, providing a range of complementary options.

For example, deep contrastive learning may be used to map the point-cloud depiction (represented as a point-cloud object in computer language) and molecular fragments into the same embedding space. The loss function comprises a dot product of a first function of the point-cloud object and a second function of the molecular fragment object. Thus, the value of the loss function becomes 1 when the point cloud matches the molecular fragment, 0 when they are completely different, and between 0 and 1 when they are partially matched. The learning process is designed to optimize the molecular fragment shape such that the value of the loss function converges to 1.

As another example, the visualization module 104 may be configured to provide intuitive and atomically precise visualization of the druggable three-dimensional space using the point-cloud representations of the pockets on the target. Unlike existing methods that primarily rely on molecular surfaces for shape depictions, this approach integrates the energy fields (e.g., non-polar interactions) within regions of the target as well as the shape of the target. This integration allows drug designers to perceive the shape and nonbonded interaction strengths as a cohesive unit, providing a more comprehensive and immediate understanding of the druggable space.

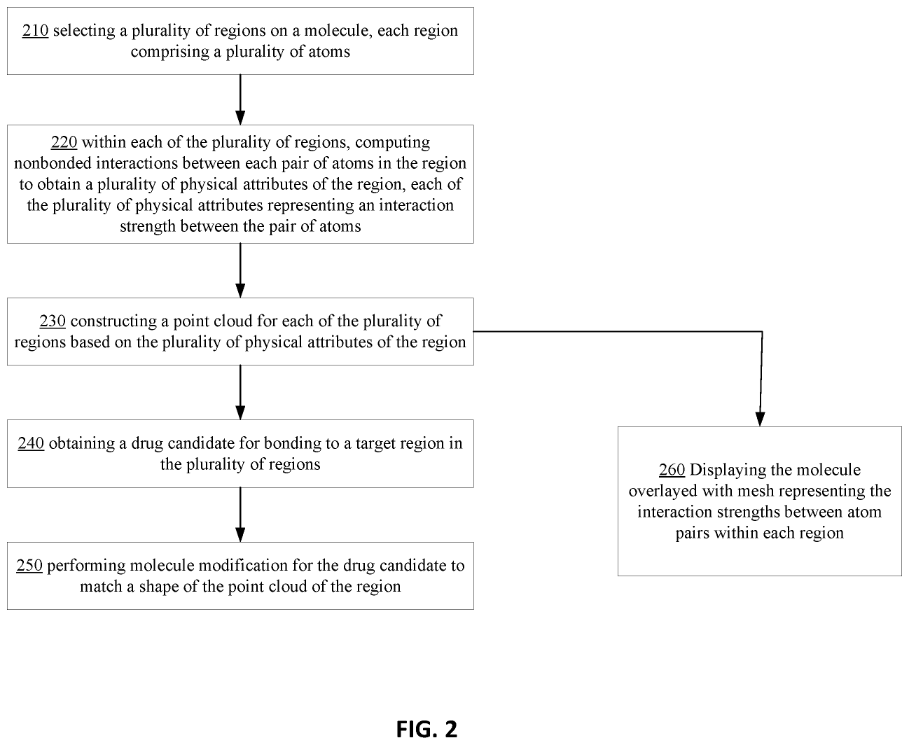

FIG. 2 illustrates an example method for using physics-based depictions of protein shapes in visualization and shape-conditioned drug candidate generation, in accordance with various embodiments. The method illustrated in FIG. 2 provides some exemplary implementation details of the system illustrated in FIG. 1. Some steps of the method in FIG. 2 may be performed by the backend server 102 or any of the downstream modules 104 and 106 in FIG. 1. The example method in FIG. 2 is applicable to drug generation, which involves the study of molecule binding. When developing a new drug, scientists aim to design molecules that can effectively bind to specific target proteins or receptors in the body. Binding interactions between a drug molecule and its target are critical for achieving the desired therapeutic effect.

In the context of molecule binding, a pocket refers to a small cavity or crevice on the surface of a protein or other molecular target. These pockets are three-dimensional regions that can accommodate the binding of specific molecules, such as ligands or substrates. Protein pockets are typically formed by the arrangement of amino acid residues within the protein's structure. The residues lining the pocket may have specific chemical and structural properties that enable them to interact with complementary molecules. These interactions can include hydrogen bonding, electrostatic interactions, van der Waals forces, and hydrophobic interactions. The shape and size of a pocket can vary, and it plays a critical role in determining the binding specificity and affinity of a molecule.

The pocket provides a binding site where the ligand or substrate can fit snugly, forming specific interactions with the residues within the pocket. The complementarity between the shape, size, and chemical properties of the pocket and the ligand or substrate is crucial for effective binding. A well-matched pocket-ligand interaction enhances the likelihood of favorable interactions and contributes to the overall stability and specificity of the molecular complex. For simplicity, the following descriptions refer the amino acid residues within a pocket as atoms. A person skilled in the art would appreciate that each amino acid residue usually includes multiple atoms (e.g., carbon, hydrogen, oxygen, nitrogen, and sometimes sulfur) bonded together in a specific arrangement.

Step 210 may include selecting a plurality of regions on a molecule (e.g., a target protein), each region including a plurality of atoms, typically on the basis of a chemically meaningful fragmentation of the organic small molecule, which is important for ensuring that the suggested modifications are synthetically accessible. The range of the selection may be adjusted based on the desired accuracy and comprehensiveness of the method. For the optimal accuracy and comprehensiveness, the entire molecule may be selected for processing. Alternatively, a subset of the regions may be selected as candidate pocket areas using ligand binding site prediction methods. These methods may use algorithms and scoring functions to predict the most probable binding sites based on factors like shape complementarity, electrostatic interactions, and hydrophobicity. Tools such as SiteMap, POCASA, and LIGSITE are often used for this purpose.

In some embodiments, the selection of the regions on the molecule may be based on a design policy manually-constructed or machine-learned to map the local point-cloud features to shape-complementary molecular fragments. For effective molecular design, the policy may consider common molecular fragments that are used in medicinal chemistry, which include heterocyclic backbones (e.g., pyrimidine, piperidine, thiazole), side-chain functional groups (e.g., —CN, —Cl, -Me, cyclopropyl), intermediate linkers (e.g., amide, ether). A preferred policy should be defined or learned to provide the optimal molecular fragment as preconditioned by the local point cloud. In some embodiments, the policy is subject to on-the-fly updates according to user feedbacks.

Step 220 may include computing, within each of the plurality of selected regions, nonbonded interactions between each pair of the plurality of atoms in the region to obtain a plurality of physics-based attributes of the region, each of the plurality of physics-based attributes representing an interaction strength between the pair of atoms. For example, Lennard-Jones potential (e.g., Lennard-Jones 12-6 potential) may be used to estimate the energy field of a region (e.g., pocket candidate) on a molecule. The Lennard-Jones potential describes the intermolecular interactions between atoms or particles and provides an approximation of the potential energy as a function of their separation distance. In some embodiments, in each selected region, the atom types in each pair of atoms may first be determined based on each atom's chemical properties. Common atom types include carbon (C), nitrogen (N), oxygen (O), and hydrogen (H). The atom types may correspond to Lennard-Jones parameters used in the potential energy calculation. Next, a pair of Lennard-Jones parameters (F and a values) may be determined or calculated for each atom type within the pocket. The epsilon (F) parameter characterizes the strength of the attractive interactions, and the sigma (a) parameter determines the distance at which the repulsive interactions become dominant. Then, the potential interaction energy/strength between each pair of atoms may be computed using 12-6 formula V(r)=4_{ε}[(σ/r)\hat{ }12−(σ/r)\hat{ }6], where V(r) is the potential energy at distance r, ε is the epsilon value, σ is the sigma value, and r is the distance between atoms. Alternative functional forms such as 8-6, 9-6, 14-7, or the universal nonbonded potential may also be considered to accurately represent the nonbonded interactions.

Step 230 may include constructing a point cloud for each of the plurality of regions based on the plurality of physics-based attributes of the region. In some embodiments, the point cloud for each of the selected regions (pocket candidates) may be constructed with the following process.

Data Preparation: Obtain the structure of the protein or target molecule in a suitable format, such as Protein Data Bank (PDB) files or other molecular file formats. This file may include information about the atomic coordinates and connectivity of the atoms in the protein or target molecule.

Point Cloud Object: Create a point cloud object for each pocket region that serves as a representation of the nonbonded interactions among the atoms within the pocket region. Each point within the point cloud corresponds to a specific attribute or physical quantity associated with a nonbonded interaction. The attributes can include the strength of Pauli repulsion, the magnitude of London dispersion forces, solvation effects, and hydrophobic interactions.

Visualization: Visualize a structure of the molecule by overlaying a mesh on the plurality of regions of the molecule based on the point cloud and associated attributes for each of the plurality of regions on a graphic user interface (GUI) at step 260. The visualization provides a better understanding of the potential nonbonded interactions between the drug and target molecules. It can aid in identifying regions of strong or weak interactions, guiding further analysis and decision-making in drug design activities.

In some embodiments, the construction of the point clouds may be selective. For example, for each candidate region, a non-polar potential field for the region may be determined based on the nonbonded interactions between the neighboring protein atoms and the nonbonded interactions between a set of probe atoms that are randomly and locally sampled in the 3D space. If the non-polar potential field being negative and having an absolute value greater than a threshold, a point cloud may be constructed for the region. Otherwise, no point cloud may be constructed to save computational resources.

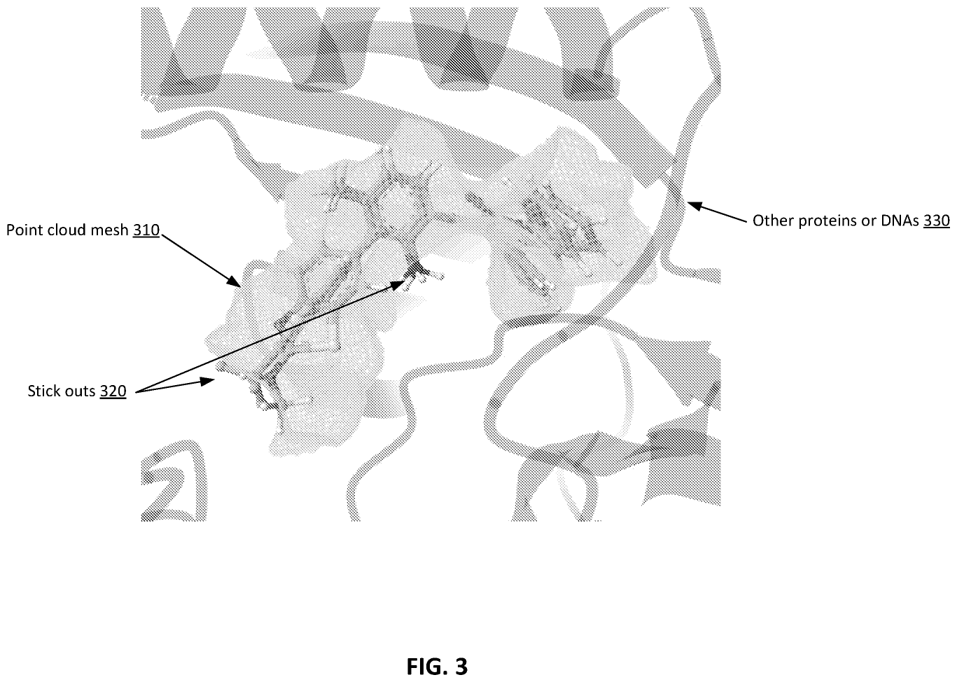

An example mesh visualization of the point cloud for a protein is illustrated in FIG. 3. As shown, the point cloud mesh 310 is visually overlayed around the protein structure, and the shape of the point cloud mesh 310 are determined based on the nonbonded interaction strengths within the regions of the protein thus reflect potential bonding strength between the protein and a drug. There may be regions 320 of the protein that stick out from the mesh. These regions either have strong polar interaction strengths or are invalid, and may be ignored in subsequent process.

Referring back to FIG. 2, after obtaining a drug candidate for bonding to a target region in the protein at step 240, a researcher is allowed to adjust the view (e.g., dragging around) of the 3D representation (including the mesh 310, the protein, and other proteins or DNAs 330) to evaluate the candidate pocket regions for bonding, and subsequently perform molecular modifications of the drug at step 250 to precisely match the shape of the point cloud. In some embodiments, the molecular modification may include scaffold modification (modifying the core structure of the ligand to better fit the shape of the pocket), side chain optimization (adjusting torsion angles, introducing or removing functional groups, or changing the length or orientation of the side chains), conformational sampling (conformational sampling on the ligand to explore different conformations and orientations within the pocket), functional group addition or removal, and/or linker optimization (adjusting the length, flexibility, or rigidity of the linker to optimize the positioning of the ligand within the pocket).

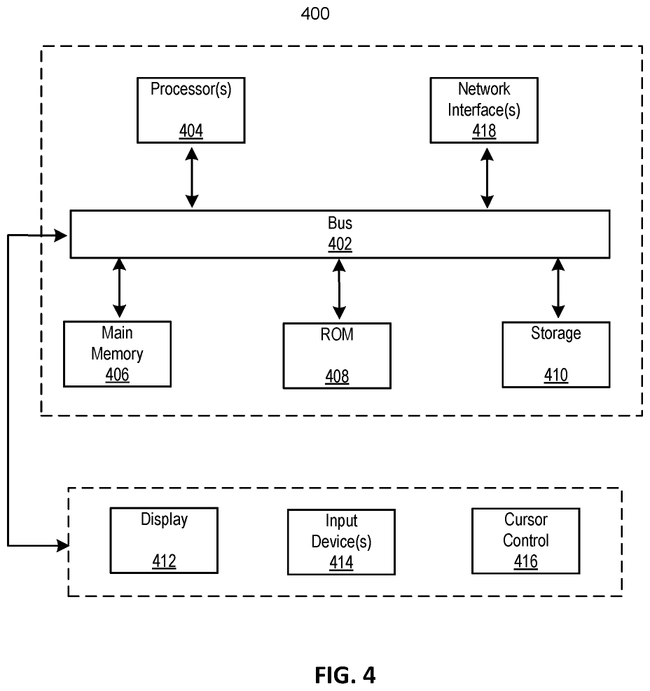

FIG. 4 illustrates a block diagram of an example computer system 400 in which any of the embodiments described herein may be implemented. The computer system 400 includes a bus 402 or other communication mechanisms for communicating information, one or more hardware processors 404 coupled with bus 402 for processing information. Hardware processor(s) 404 may be, for example, one or more general-purpose microprocessors.

The computer system 400 also includes a main memory 406, such as a random access memory (RAM), cache and/or other dynamic storage devices, coupled to bus 402 for storing information and instructions to be executed by processor 404. Main memory 406 also may be used for storing temporary variables or other intermediate information during execution of instructions to be executed by processor 404. Such instructions, when stored in storage media accessible to processor 404, render computer system 400 into a special-purpose machine that is customized to perform the operations specified in the instructions.

The computer system 400 further includes a read-only memory (ROM) 408 or other static storage device coupled to bus 402 for storing static information and instructions for processor 404. A storage device 410, such as a magnetic disk, optical disk, or USB thumb drive (Flash drive), etc., is provided and coupled to bus 402 for storing information and instructions.

The computer system 400 may be coupled via bus 402 to a display 412, such as a cathode ray tube (CRT) or LCD display (or touch screen), for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. This input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

The computing system 400 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage.

The computer system 400 may implement the techniques described herein using customized hard-wired logic, one or more ASICs or FPGAs, firmware and/or program logic which in combination with the computer system causes or programs computer system 400 to be a special-purpose machine. According to one embodiment, the techniques herein are performed by computer system 400 in response to processor(s) 404 executing one or more sequences of one or more instructions contained in main memory 406. Such instructions may be read into main memory 406 from another storage medium, such as storage device 410. Execution of the sequences of instructions contained in main memory 406 causes processor(s) 404 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 410. Volatile media includes dynamic memory, such as main memory 406. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 402. Transmission media can also take the form of acoustic or light waves, such as those generated during radio-wave and infra-red data communications.

Various forms of media may be involved in carrying one or more sequences of one or more instructions to processor 404 for execution. For example, the instructions may initially be carried on a magnetic disk or solid state drive of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may retrieves and executes the instructions. The instructions received by main memory 406 may optionally be stored on storage device 410 either before or after execution by processor 404.

The computer system 400 also includes a communication interface 418 coupled to bus 402. Communication interface 418 provides a two-way data communication coupling to one or more network links that are connected to one or more local networks. For example, communication interface 418 may be an integrated services digital network (ISDN) card, cable modem, satellite modem, or a modem to provide a data communication connection to a corresponding type of telephone line. As another example, communication interface 418 may be a local area network (LAN) card to provide a data communication connection to a compatible LAN (or WAN component to communicated with a WAN). Wireless links may also be implemented. In any such implementation, communication interface 418 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet”. Local network and Internet both use electrical, electromagnetic or optical signals that carry digital data streams. The signals through the various networks and the signals on network link and through communication interface 418, which carry the digital data to and from computer system 400, are example forms of transmission media.

The computer system 400 can send messages and receive data, including program code, through the network(s), network link and communication interface 418. In the Internet example, a server might transmit a requested code for an application program through the Internet, the ISP, the local network and the communication interface 418.

The received code may be executed by processor 404 as it is received, and/or stored in storage device 410, or other non-volatile storage for later execution.

Each of the processes, methods, and algorithms described in the preceding sections may be embodied in, and fully or partially automated by, code modules executed by one or more computer systems or computer processors comprising computer hardware. The processes and algorithms may be implemented partially or wholly in application-specific circuitry.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be removed, executed out of order from that shown or discussed, including substantially concurrently or in reverse order, depending on the functionality involved, as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

### Language

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the subject matter has been described with reference to specific example embodiments, various modifications and changes may be made to these embodiments without departing from the broader scope of embodiments of the present disclosure. Such embodiments of the subject matter may be referred to herein, individually or collectively, by the term “invention” merely for convenience and without intending to voluntarily limit the scope of this application to any single disclosure or concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

It will be appreciated that an “engine,” “system,” “data store,” and/or “database” may comprise software, hardware, firmware, and/or circuitry. In one example, one or more software programs comprising instructions capable of being executable by a processor may perform one or more of the functions of the engines, data stores, databases, or systems described herein. In another example, circuitry may perform the same or similar functions. Alternative embodiments may comprise more, less, or functionally equivalent engines, systems, data stores, or databases, and still be within the scope of present embodiments. For example, the functionality of the various systems, engines, data stores, and/or databases may be combined or divided differently.

“Open source” software is defined herein to be source code that allows distribution as source code as well as compiled form, with a well-publicized and indexed means of obtaining the source, optionally with a license that allows modifications and derived works.

The data stores described herein may be any suitable structure (e.g., an active database, a relational database, a self-referential database, a table, a matrix, an array, a flat file, a documented-oriented storage system, a non-relational No-SQL system, and the like), and may be cloud-based or otherwise.

As used herein, the term “or” may be construed in either an inclusive or exclusive sense. Moreover, plural instances may be provided for resources, operations, or structures described herein as a single instance. Additionally, boundaries between various resources, operations, engines, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

Although the invention has been described in detail for the purpose of illustration based on what is currently considered to be the most practical and preferred implementations, it is to be understood that such detail is solely for that purpose and that the invention is not limited to the disclosed implementations, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment. A component being implemented as another component may be construed as the component being operated in a same or similar manner as the another component, and/or comprising same or similar features, characteristics, and parameters as the another component.

The phrases “at least one of,” “at least one selected from the group of,” or “at least one selected from the group consisting of,” and the like are to be interpreted in the disjunctive (e.g., not to be interpreted as at least one of A and at least one of B).

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment, but may be in some instances. Furthermore, the particular features, structures, or characteristics may be combined in any suitable manner in one or more embodiments.

1. A computer-implemented method, comprising:
selecting a plurality of regions on a molecule, each region comprising a plurality of atoms;
within each of the plurality of regions, computing nonbonded interactions between each pair of the plurality of atoms in the region to obtain a plurality of physical attributes of the region, each of the plurality of physical attributes representing an interaction strength between the pair of atoms;
constructing a point cloud for each of the plurality of regions based on the plurality of physical attributes of the region;
obtaining a drug candidate for bonding to a target region in the plurality of regions; and
performing molecule modification on the drug candidate to match a shape of the point cloud of the target region.

2. The computer-implemented method of claim 1, further comprising:
displaying, on a graphic user interface (GUI), a structure of the molecule by overlaying mesh on the plurality of regions based on the point cloud for each of the plurality of regions.

3. The computer-implemented method of claim 2, wherein the displaying comprises, for each of the plurality of regions on the molecule:
performing quantization on the plurality of attributes of the region into a plurality of strength values; and
generating a mesh for the region that comprises a plurality of vertices corresponding to the plurality of attributes, wherein coordinates of the plurality of vertices are determined based on the plurality of strength values.

4. The computer-implemented method of claim 2, wherein the displaying comprises, for each of the plurality of regions on the molecule:
performing quantization on the plurality of attributes of the region into a plurality of strength values; and
generating a mesh for the region by defining color gradients on a face of the mesh based on the plurality of strength values.

5. The computer-implemented method of claim 1, wherein the plurality of physical attributes include:
quantified repulsion;
quantified dispersion attractions,
quantified solvation; or
quantified hydrophobic effect.

6. The computer-implemented method of claim 1, wherein the computing nonbonded interactions between each pair of atoms in the region uses an Lennard-Jones potential.

7. The computer-implemented method of claim 1, wherein the computing nonbonded interactions between each pair of atoms in the region to obtain a plurality of physical attributes of the region comprises:
determining atom types of the pair of atoms based on each atom's chemical properties;
determining a first parameter characterizing a strength of an attractive interaction between the pair of atoms;
determining a second parameter representing a distance at which a repulsive interaction between the pair of atoms is dominant; and
computing a potential interaction strength between the pair of atoms using the first parameter and the second parameter.

8. The computer-implemented method of claim 1, wherein the molecule modification for the drug candidate comprises:
scaffold modification;
side chain optimization;
conformational sampling;
functional group addition or removal; or
linker optimization.

9. The computer-implemented method of claim 1, further comprising:
determining a non-polar potential field for the region based on the nonbonded interactions between each pair of the plurality of atoms in the region;
wherein the constructing the point cloud for the region comprises:
in response to the non-polar potential field being negative and having an absolute value greater than a threshold, constructing the point cloud for the region.

10. A computing system, comprising:
one or more processors; and
a memory storing instructions that, when executed by the one or more processors, cause the system to perform operations comprising:
selecting a plurality of regions on a molecule, each region comprising a plurality of atoms;
within each of the plurality of regions, computing nonbonded interactions between each pair of the plurality of atoms in the region to obtain a plurality of physical attributes of the region, each of the plurality of physical attributes representing an interaction strength between the pair of atoms;
constructing a point cloud for each of the plurality of regions based on the plurality of physical attributes of the region;
obtaining a drug candidate for bonding to a target region in the plurality of regions; and
performing molecule modification on the drug candidate to match a shape of the point cloud of the target region.

11. The computing system of claim 10, wherein the operations further comprise:
determining a non-polar potential field for the region based on the nonbonded interactions between each pair of the plurality of atoms in the region;
wherein the constructing the point cloud for the region comprises:
in response to the non-polar potential field being negative and having an absolute value greater than a threshold, constructing the point cloud for the region.

12. The computing system of claim 10, wherein the operations further comprise:
displaying, on a graphic user interface (GUI), a structure of the molecule by overlaying mesh on the plurality of regions based on the point cloud for each of the plurality of regions.

13. The computing system of claim 12, wherein the displaying comprises, for each of the plurality of regions on the molecule:
performing quantization on the plurality of attributes of the region into a plurality of strength values; and
generating a mesh for the region by assigning the plurality of strength values to corresponding vertices of the mesh.

14. The computing system of claim 12, wherein the displaying comprises, for each of the plurality of regions on the molecule:
performing quantization on the plurality of attributes of the region into a plurality of strength values; and
generating a mesh for the region by defining color gradients on a face of the mesh based on the plurality of strength values.

15. The computing system of claim 10, wherein the plurality of physical attributes include:
quantified repulsion;
quantified dispersion attractions,
quantified solvation; or
quantified hydrophobic effect.

16. The computing system of claim 10, wherein the computing nonbonded interactions between each pair of atoms in the region to obtain a plurality of physical attributes of the region comprises:
determining atom types of the pair of atoms based on each atom's chemical properties including carbon, oxygen, or nitrogen;
determining a first parameter characterizing a strength of an attractive interaction between the pair of atoms;
determining a second parameter representing a distance at which a repulsive interaction between the pair of atoms is dominant; and
computing a potential interaction strength between the pair of atoms using the first parameter and the second parameter.

17. The computing system of claim 10, wherein the molecule modification for the drug candidate comprises:
scaffold modification;
side chain optimization;
conformational sampling;
functional group addition or removal; or
linker optimization.

18. A non-transitory computer-readable storage medium configured with instructions executable by one or more processors to cause the one or more processors to perform operations comprising:
selecting a plurality of regions on a molecule, each region comprising a plurality of atoms;
within each of the plurality of regions, computing nonbonded interactions between each pair of the plurality of atoms in the region to obtain a plurality of physical attributes of the region, each of the plurality of physical attributes representing an interaction strength between the pair of atoms;
constructing a point cloud for each of the plurality of regions based on the plurality of physical attributes of the region;
obtaining a drug candidate for bonding to a target region in the plurality of regions; and
performing molecule modification on the drug candidate to match a shape of the point cloud of the target region.

19. The non-transitory computer-readable storage medium of claim 18, wherein the operations further comprise:
determining a non-polar potential field for the region based on the nonbonded interactions between each pair of the plurality of atoms in the region;
wherein the constructing the point cloud for the region comprises:
in response to the non-polar potential field being negative and having an absolute value greater than a threshold, constructing the point cloud for the region.

20. The non-transitory computer-readable storage medium of claim 18, wherein the operations further comprise:
displaying, on a graphic user interface (GUI), a structure of the molecule by overlaying mesh on the plurality of regions based on the point cloud for each of the plurality of regions, wherein the displaying comprises:
performing quantization on the plurality of attributes of the region into a plurality of strength values; and
generating a mesh for the region by assigning the plurality of strength values to corresponding vertices of the mesh.
